(19) United States
(12) Patent Application Publication
(10) Pub. No.: US 2024/0242342 A1
(43) Pub. Date: Jul. 18, 2024

(54) APPARATUS AND METHOD FOR AUTOMATED ANALYSIS OF LOWER EXTREMITY IMAGE

(71) Applicant: CONNECTEVE CO., Ltd., Seoul (KR)

(72) Inventor: Du Hyun RO, Seoul (KR)

(73) Assignee: CONNECTEVE CO., Ltd., Seoul (KR)

(21) Appl. No.: 18/415,400

(22) Filed: Jan. 17, 2024

(30) Foreign Application Priority Data
Jan. 17, 2023 (KR) 10-2023-0006749

(51) Int. Cl. G06T 7/00; G06T 3/40; G06T 7/60; G16H 30/40

(52) U.S. Cl. CPC G06T 7/0012; G06T 3/40; G06T 7/60; G16H 30/40; G06T 2207/20044; G06T 2207/30008; G06T 2207/30204

(57) ABSTRACT

Provided are an apparatus, a method, and a system for automated analysis of knee joint space in a lower extremity image, the apparatus comprising: a processor; and a memory including one or more instructions implemented to be executed by the processor, wherein the processor generates a pre-lower extremity image by preprocessing an original lower extremity image from a camera; identifies a plurality of anatomical landmarks in the pre-lower extremity image based on a machine learning model; generates the lower extremity image in which a position of the anatomical landmark is identified by processing the pre-lower extremity image; and derives a width of the knee joint space in the lower extremity image using the position of the anatomical landmark.

### A. TECHNICAL FIELD

The present disclosure relates to an automated analysis of a lower extremity image, more particularly, to an apparatus and method for automatically analyzing a joint space width of the lower extremity image using a machine learning model.

### B. DESCRIPTION OF THE RELATED ART

With development of artificial intelligence learning models, many machine learning models are being used to read medical images. For example, the machine learning models such as Convolutional Neural Networks (CNN), Deep Neural Networks (DNN), Recurrent Neural Networks (RNN), and Deep Belief Networks (DBN) are being applied to detect, classify, and characterize the medical images.

The machine learning models are currently being used to support an image reading, an image finding, an image diagnosis to predict a disease of a patient. More specifically, a method of supporting the image reading, the image finding, and the image diagnosis of the medical image is to obtain the biometric image from the patient, extract feature from the fundus image based on the machined learning models, provide the feature to a practitioner, and predict the patient's disease based on it. In this case, the feature includes various information for the medical image.

Meanwhile, the analysis of lower extremity images is a critical process in the diagnosis and treatment of knee osteoarthritis patients. However, this process is repetitive and time-consuming, making it difficult for physicians to perform it manually. Additionally, there is a problem that errors can occur in the measurement of joint space width depending on the posterior tibial slope angle and beam projection angle.

Machine learning models or deep learning models can be used to solve these problems. Machine learning models can learn from large amounts of data to recognize certain patterns. Therefore, they can automatically recognize the characteristics of the knee joint in lower extremity images and accurately measure the joint space width based on them. Additionally, developing algorithms that can compensate for the difference between the posterior tibial slope angle (PTSA) and beam projection angle (BPA) can reduce these errors.

### SUMMARY OF THE DISCLOSURE

In one aspect of the present disclosure, an apparatus for automated analysis of knee joint space in a lower extremity image includes a processor; and a memory including one or more sequences of instructions which, when executed by the processor, causes steps to be performed comprising: generating a pre-lower extremity image by preprocessing an original lower extremity image from a camera; identifying a plurality of anatomical landmarks in the pre-lower extremity image based on a machine learning model; generating the lower extremity image in which a position of the anatomical landmark is identified by processing the pre-lower extremity image; and deriving a width of the knee joint space in the lower extremity image using the position of the anatomical landmark.

Desirably, the steps further may include generating images in which a specific anatomical area of the lower extremity image is enlarged; and displaying the images on a display device.

Desirably, the images may include at least one of a hip joint image, a knee joint image and an ankle joint image.

Desirably, the steps further may include generating a marker indicating the width of knee joint space on the lower extremity image; and displaying the lower extremity image including the marker on a display device.

Desirably, the position of the anatomical landmark may include at least one of a medial or lateral femoral condyle, a medial or lateral anterior border of tibia measured from a midline of the medial plateau or a center point of the medial plateau, a medial or lateral posterior border of tibia measured from the center point of the medial plateau.

Desirably, the width of the knee joint space may be derived by calculating an average of a first distance and a second distance, wherein the first distance may be a distance between a medial or lateral femoral condyle and a medial or lateral anterior border of tibia measured from a midline of a medial plateau or a center point of the medial plateau, and the second distance may be a distance between the medial or lateral femoral condyle and a medial or lateral posterior border of tibia, measured from the center point of the medial plateau.

In another aspect of the present disclosure, a method for automated analysis of knee joint space in a lower extremity image includes generating a pre-lower extremity image by preprocessing an original lower extremity image from a camera; identifying a plurality of anatomical landmarks in the pre-lower extremity image based on a machine learning model; generating the lower extremity image in which a position of the anatomical landmark is identified by processing the pre-lower extremity image; and deriving a width of the knee joint space in the lower extremity image using the position of the anatomical landmark.

### BRIEF DESCRIPTION OF THE DRAWINGS

FIG. 1 is a schematic diagram of an illustrative apparatus for automated analysis of a lower extremity image according to embodiments of the present disclosure.

FIG. 2 is a schematic diagram of an illustrative system for automated analysis of a lower extremity image according to embodiments of the present disclosure.

FIG. 3 is a view showing an illustrative process for generating a pre-lower extremity image by a processor according to embodiments of the present disclosure.

FIG. 4 is a view showing an illustrative processing for generating a lower extremity image in which an anatomical landmark is identified by a processor according to embodiments of the present disclosure.

FIG. 5 is a view showing specific parts of an anatomical landmark marked in a lower extremity image by a processor according to embodiments of the present disclosure.

FIG. 6A to 6C are views showing an illustrative processing for deriving width of a knee joint space on a lower extremity image by processor according to embodiments of the present disclosure.

FIG. 7 shows the Kellgren-Lawrence grading scale used to experiment with the performance of an apparatus 100 according to embodiments of the present disclosure.

FIG. 8 is a graph showing the correlation between the first distance, the second distance, and the average distance analyzed by an apparatus according to embodiments of the present disclosure.

FIG. 9 is a graph showing a difference between an average distance at different beam projection angles (BPA) and an actual joint space width (JSW) analyzed by an apparatus according to embodiments of the present disclosure.

FIG. 10 shows a flowchart illustrating an exemplary process for automated analysis of a lower extremity image by a processor 111 according to embodiments of the present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details.

Furthermore, one skilled in the art will recognize that embodiments of the present disclosure, described below, may be implemented in a variety of ways, such as a process, an apparatus, a system, a device, or a method on a tangible computer-readable medium.

The terms “comprise/include” used throughout the description and the claims and modifications thereof are not intended to exclude other technical features, additions, components, or operations.

In the following description, it shall also be noted that the terms “learning” shall be understood not to intend mental action such as human educational activity because of referring to performing machine learning by a processing module such as a processor, a CPU, an application processor, micro-controller, so on.

An “image” is defined as a reproduction or imitation of the form of a person or thing, or specific characteristics thereof, in digital form. An image can be, but is not limited to, a JPEG image, a PNG image, a GIF image, a TIFF image, or any other digital image format known in the art. “Image” is used interchangeably with “photograph”.

The embodiments described herein relate generally to diagnostic medical images. Although any type of medical image can be used, these embodiments will be illustrated in conjunction with bond joint images. However, the disclosed methods, systems, apparatuses, and devices can also be used with medical images of which can support the diagnosis of a disease condition.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof. Furthermore, one skilled in the art shall recognize that certain steps may optionally be performed that steps may not be limited to the specific order set forth herein, and that certain steps may be performed in different orders, including being done contemporaneously. Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments. Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

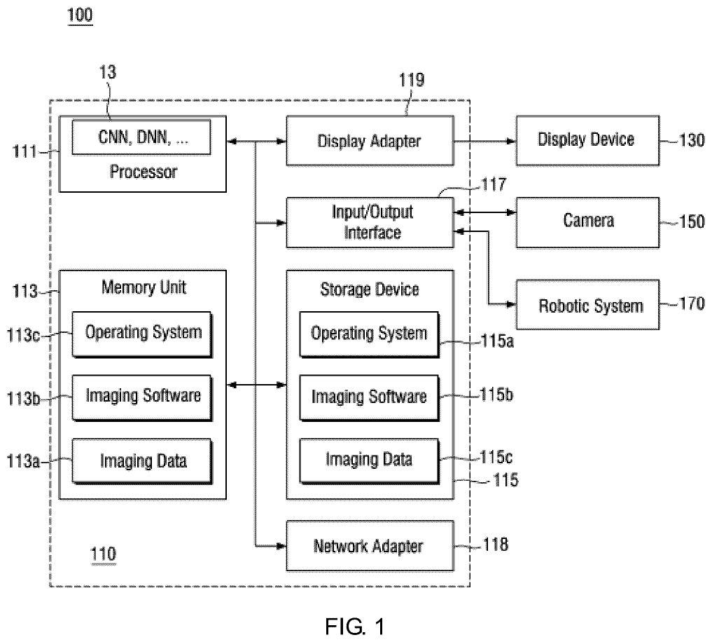

FIG. 1 is a schematic diagram of an illustrative apparatus for automated analysis of a lower extremity image according to embodiments of the present disclosure.

As depicted, the apparatus 100 may include a computing device 110, a display device 130, a camera 150 and a robotic device 170. In embodiments, the computing device 110 may include, but is not limited thereto, one or more processor 111, a memory unit 113, a storage device 115, an input/output interface 117, a network adapter 118, a display adapter 119, and a system bus 112 connecting various system components to the memory unit 113. In embodiments, the apparatus 100 may further include communication mechanisms as well as the system bus 112 for transferring information.

In embodiments, the communication mechanisms or the system bus 112 may interconnect the processor 111, a computer-readable medium, a short range communication module (e.g., a Bluetooth, a NFC), the network adapter 118 including a network interface or mobile communication module, the display device 130 (e.g., a CRT, a LCD, etc.), an input device (e.g., a keyboard, a keypad, a virtual keyboard, a mouse, a trackball, a stylus, a touch sensing means, etc.) and/or subsystems.

In embodiments, the processor 111 is, but is not limited to, a processing module, a Computer Processing Unit (CPU), an Application Processor (AP), a microcontroller, a digital signal processor.

In embodiments, the processor 111 may include an image filter such as a high pass filter or a low pass filter to filter a specific factor in a lower limb image. In addition, the processor 111 may communicate with a hardware controller such as the display adapter 119 to display a user interface on the display device 130.

In embodiments, the processor 111 may access the memory unit 113 and execute commands stored in the memory unit 113 or one or more sequences of instructions to control the operation of the apparatus 100.

The commands or sequences of instructions may be read in the memory unit 113 from computer-readable medium or media such as a static storage or a disk drive but is not limited thereto. In alternative embodiments, a hard-wired circuitry which is equipped with a hardware in combination with software commands may be used. The hard-wired circuitry can replace the soft commands. The instructions may be an arbitrary medium for providing the commands to the processor 111 and may be loaded into the memory unit 113.

In embodiments, the system bus 112 may represent one or more of several possible types of bus structures, including a memory bus or memory controller, a peripheral bus, an accelerated graphics port, and a processor or local bus using any of a variety of bus architectures. For instance, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like.

In embodiments, the system bus 112, and all buses specified in this description can also be implemented over a wired or wireless network connection.

A transmission media including wires of the system bus 112 may include at least one of coaxial cables, copper wires, and optical fibers. For instance, the transmission media may take a form of sound waves or light waves generated during radio wave communication or infrared data communication.

In embodiments, the apparatus 100 may transmit or receive the commands including messages, data, and one or more programs, i.e., a program code, through a network link or the network adapter 118.

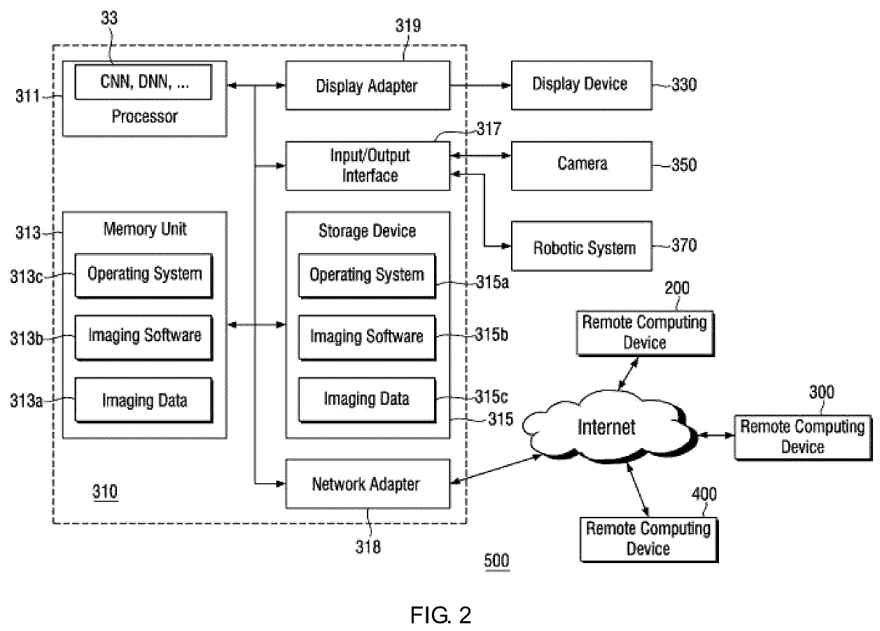

In embodiments, the network adapter 118 may include a separate or integrated antenna for enabling transmission and reception through the network link. The network adapter 118 may access a network and communicate with a remote computing devices 200, 300, 400 in FIG. 2.

In embodiments, the network may be, but is not limited to, at least one of LAN, WLAN, PSTN, and cellular phone networks. The network adapter 118 may include at least one of a network interface and a mobile communication module for accessing the network. In embodiments, the mobile communication module may be accessed to a mobile communication network for each generation such as 2G to 5G mobile communication network.

In embodiments, on receiving a program code, the program code may be executed by the processor 111 and may be stored in a disk drive of the memory unit 113 or in a non-volatile memory of a different type from the disk drive for executing the program code.

In embodiments, the computing device 110 may include a variety of computer-readable medium or media. The computer-readable medium or media may be any available medium or media that are accessible by the computing device 100. For example, the computer-readable medium or media may include, but is not limited to, both volatile and non-volatile media, removable or non-removable media.

In embodiments, the memory unit 113 may store a driver, an application program, data, and a database for operating the apparatus 100 therein. In addition, the memory unit 113 may include a computer-readable medium in a form of a volatile memory such as a random-access memory (RAM), a non-volatile memory such as a read only memory (ROM), and a flash memory. For instance, it may be, but is not limited to, a hard disk drive, a solid-state drive, an optical disk drive. In embodiments, each of the memory unit 113 and the storage device 115 may be program modules such as the imaging software 113b, 115b and the operating systems 113c, 115c that can be immediately accessed so that a data such as the imaging data 113a, 115a is operated by the processor 111.

In embodiments, the machine learning model 13 may be installed into at least one of the processors 111, the memory unit 113 and the storage device 115. The machine learning model 13 may be, but is not limited to, at least one of a deep neural network (DNN), a convolutional neural network (CNN) and a recurrent neural network (RNN), which are one of the machine learning algorithms.

The Deep Neural Network (DNN) is an Artificial Neural Network (ANN) composed of multiple hidden layers between the input layer and the output layer. Like a conventional artificial neural network, the DNN can model complex nonlinear relationships. For example, in the structure of the DNN for object recognition, each object can be represented hierarchically as a composition of basic elements in an image. Additional layers can gradually aggregate the features of sublayers. This characteristic of deep neural networks allows them to model complex data with fewer units (nodes) compared to similarly performing artificial neural networks.

The Convolutional Neural Networks (CNNs) are a type of multilayer perceptron designed to require minimal preprocessing. The CNNs consist of one or more convolutional layers followed by conventional artificial neural network layers, with additional utilization of weights and pooling layers. Thanks to this structure, the CNNs can effectively utilize 2D input data. Compared to other deep learning architectures, the CNNs perform well in both image and speech processing fields. The CNNs can also be trained using standard backpropagation, and they are easier to train and use fewer parameters than other feedforward artificial neural network techniques.

In deep learning, Convolutional Deep Belief Networks (CDBNs) have been developed. The CDBNs are structurally like traditional CNNs, allowing for effective use of 2D structures while benefiting from pretraining in Deep Belief Networks (DBNs). The CDBNs provide a common structure that can be used in various image and signal processing techniques and have been used in benchmarking results with standard image datasets like CIFAR.

The Recurrent Neural Networks (RNNs) refer to neural networks in which the connections between units form a directed cycle. The RNNs can use internal memory within the network to process arbitrary inputs. Due to this feature, the RNNs are applied in fields such as handwriting recognition and exhibit high recognition rates.

In embodiments, the camera 150 may include an image sensor (not shown) that captures an image of a subject and photoelectrically converts the image into an image signal and may photograph a lower limb image of the subject using the image sensor. The photographed lower limb image may be stored in the memory unit 113 or the storage device 115 or may be provided to the processor 111 through the input/output interface 117 and processed based on the machine learning model 13.

FIG. 2 is a schematic diagram of an illustrative system 500 for automated analysis of a lower extremity image according to embodiments of the present disclosure.

Referring to FIG. 2, the system 500 may include a computing device 310 and one and more remote computing devices 200, 300, 400. In embodiments, the computing device 310 and the remote computing devices 200, 300, 400 may be connected to each other through a network. The components 310, 311, 312, 313, 315, 317, 318, 319, 330 of the system 500 are similar to their counterparts in FIG. 1. In embodiments, each of remote computing devices 200, 300, 400 may be similar to the apparatus 100 in FIG. 1. For instance, each of remote computing devices 200, 300, 400 may include each of the subsystems, including the processor 311, the memory unit 313, an operating system 313c, 315a, an imaging software 313b, 315b, an imaging data 313a, 315c, a network adapter 318, a storage device 315, an input/output interface 317 and a display adapter 319. Each of remote computing devices 200, 300, 400 may further include a display device 330 and a camera 350. In embodiments, the system bus 312 may connect the subsystems to each other.

In embodiments, the computing device 310 and the remote computing devices 200, 300, 400 may be configured to perform one or more of the methods, functions, and/or operations presented herein. Computing devices that implement at least one or more of the methods, functions, and/or operations described herein may comprise an application or applications operating on at least one computing device. The computing device may comprise one or more computers and one or more databases. The computing device may be a single device, a distributed device, a cloud-based computer, or a combination thereof.

It shall be noted that the present disclosure may be implemented in any instruction-execution/computing device or system capable of processing data, including, without limitation laptop computers, desktop computers, and servers. The present invention may also be implemented into other computing devices and systems. Furthermore, aspects of the present invention may be implemented in a wide variety of ways including software (including firmware), hardware, or combinations thereof. For example, the functions to practice various aspects of the present invention may be performed by components that are implemented in a wide variety of ways including discrete logic components, one or more application specific integrated circuits (ASICs), and/or program-controlled processors. It shall be noted that the way these items are implemented is not critical to the present invention.

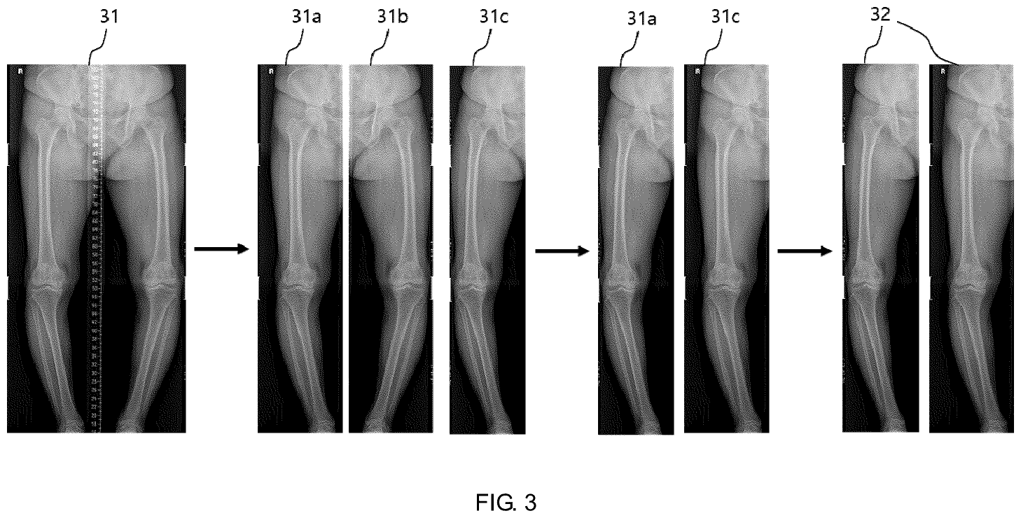

FIG. 3 is a view showing an illustrative process for generating a pre-lower extremity image by a processor according to embodiments of the present disclosure.

Referring to FIG. 3, on an original lower extremity image captured by camera, the processor 111 in FIG. 1 first determines whether the original lower extremity image 31 includes both a left leg image and a right leg image. As a result of the determination, if the original lower extremity image 31 includes both a left leg image and a right leg image, the processor 111 may divide the original lower extremity image 31 into a left leg image 31b and a right leg image 31a and inverts the left or right of the divided left leg image 31b or right leg image 31a. For example, the right leg image 31a is left as it is, and the left leg image 31b is inverted to generate a left leg image 31c with the left and right reversed.

After that, the processor 111 may adjust at least one of the brightness, contrast, saturation, and size of the left and right leg images and remove noise to generate a pre-lower extremity image 32.

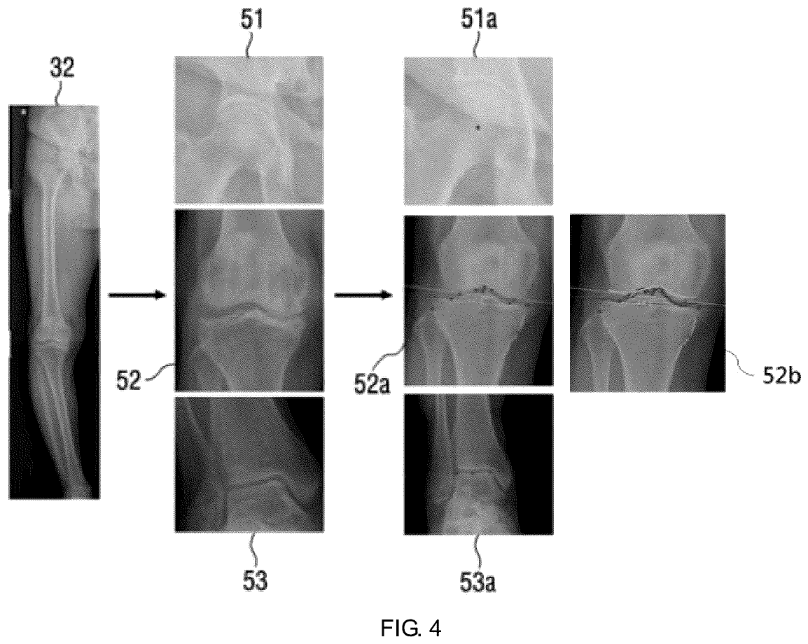

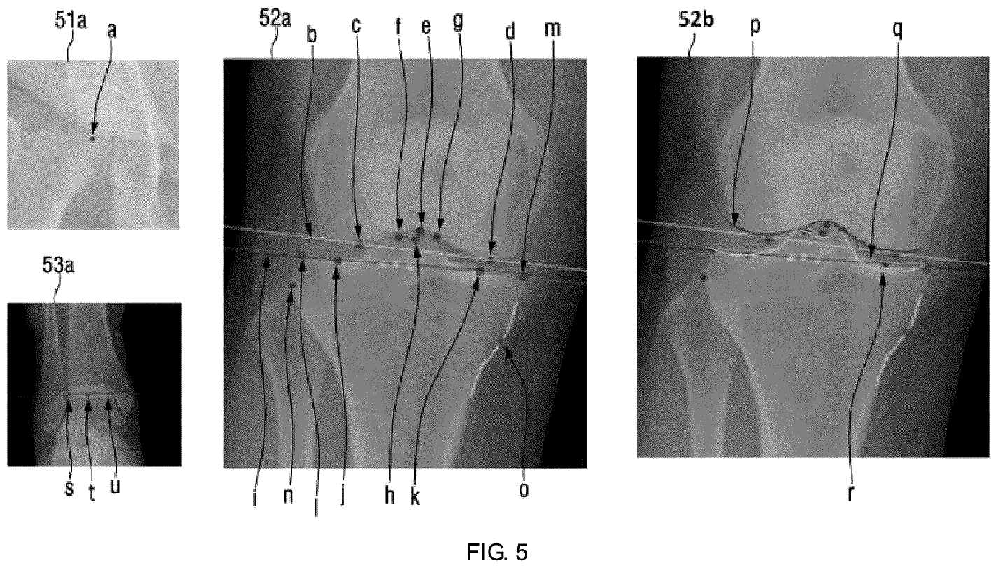

FIG. 4 is a view showing an illustrative processing for generating a lower extremity image in which an anatomical landmark is identified by a processor according to embodiments of the present disclosure and FIG. 5 is a view showing specific parts of an anatomical landmark marked in a lower extremity image by a processor according to embodiments of the present disclosure.

Referring to FIG. 4, the processor 111 may identify each joint in the pre-lower extremity image 32 using a machine learning model. In this case, the processor 111 may generate images 51, 52, 53 by processing the pre-lower extremity image 32 so that a specific region of the pre-lower extremity image is enlarged to confirm a feature information more intensively on the pre-lower extremity image 32.

In embodiments, the feature information may be, but is not limited to, the width of the joint space, bone deformities, and bone fractures. In embodiments, the images 51, 52, 53 may include images of the hip joint, knee joint, and ankle joint. The processor 111 may then generate new images 51a, 52a, 52b, 53a with the anatomical landmarks on each of the images 51, 52, 53 highlighted. For example, as depicted in FIG. 5, the processor 111 may use a machine learning model to identify the center of the femoral head (a) in the image 51a of the hip joint. It then may generate the new image 51a of the hip joint with the center of the femoral head (a) marked. The processor 111 may also use a machine learning model to identify the following anatomical landmarks in images 52a, 52b of the knee joint: Joint line (b), Two contact points (c, d) where the joint line (b) meets the femoral condyle, Femoral notch or intercondylar fossa of femur (e), Lateral/medial spine of tibia or intercondylar tubercle (f, g), Intercondylar area (h), Tibial plateau line (i), Medial/lateral midpoint of the tibial plateau line (j, k), Medial/lateral edge of the tibia (l, m), Osteotomy insertion point (n, o), Border of femur (p), Posterior border of tibia (q), Anterior border of tibia (r). The processor 111 then may generate the images 52a, 52b of the knee joint, one with all of the identified points and lines marked, and one with just the central points marked. The processor 111 may also use a machine learning model to identify the following anatomical landmarks in the image 53 of the ankle joint: Lateral edge of the talar dome (s), Center of the talar dome (t), Medial edge of the talar dome (u). The processor 111 then may generate the image 53a of the ankle joint with the identified points marked.

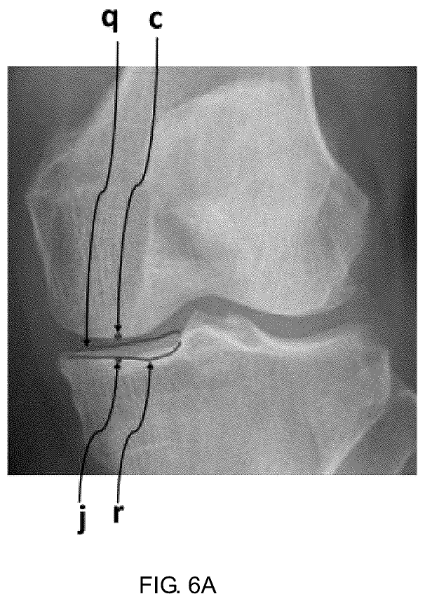

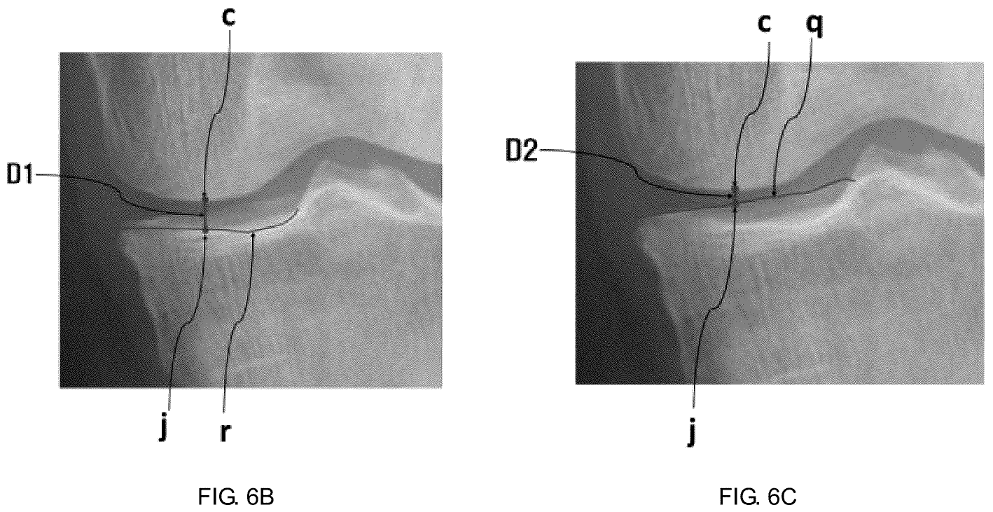

FIG. 6A to 6C are views showing an illustrative processing for deriving width of a knee joint space on a lower extremity image by processor according to embodiments of the present disclosure.

Referring to FIGS. 6A to 6C, first the processor 111 may use a machine learning model to identify anatomical landmarks of the tibia in the high-resolution image of the knee joint, such as the medial or lateral anterior border of tibia (r), the medial or lateral posterior border of tibia (q), or the medial or lateral midpoint of tibial plateau (j). The processor also may identify anatomical landmarks of the femur in the high-resolution image, such as the medial or lateral femoral condyle (c). Then, the processor may generate the high-resolution image of the tibia and femur around the knee joint with the recognized points and lines marked.

After that, the processor 111 may identify the positions of multiple anatomical landmarks in the knee joint using a machine learning model and then may calculate two distances between the tibia and femur using these positions. For example, a distance 1 (D1) is the distance between the medial (or lateral) femoral condyle (c) and the medial (or lateral) anterior border of tibia (r), measured from the midline of the medial plateau (or the center point of the medial plateau), and a distance 2 (D2) is the distance between the medial (or lateral) femoral condyle (c) and the medial (or lateral) posterior border of tibia (q), measured from the center point (j) of the medial plateau.

The processor 111 then may calculate the average of these two distances, which is used to determine the joint space width. To obtain more accurate results, the beam projection angle (BPA) may be caudal tilting rather than cephalic tilting, and it may be between 5° and 10°.

Thus, the processor 111 may automatically measure the joint space width in a knee joint image such as X-ray image. The processor may use a machine learning model to identify the positions of multiple anatomical structures in the image. The model may be trained on a dataset of X-ray images with known joint space widths.

The processor 111 may be designed to be more accurate than manual measurement. Manual measurement is subject to human error, such as misidentification of anatomical structures or inaccurate measurements. As such, the process may be also more efficient than manual measurement. Manual measurement can be time-consuming, especially for complex cases. The process can be used for a variety of purposes, such as medical diagnosis, research, and education. For example, the joint space width can be used to diagnose joint diseases, such as osteoarthritis or rheumatoid arthritis. The joint space width also can be used to study the development of joint diseases or to track the progress of treatment. The joint space width also can be used to create educational materials that help people learn about joint diseases.

In this way, the processor 111 may pre-process the original lower extremity image obtained from the camera to generate a pre-lower extremity image. Then, it can recognize multiple anatomical landmarks in the pre-lower extremity image based on a machine learning model. Next, it can display the positions of the anatomical landmarks to generate a lower extremity image. Next, it can also derive the knee joint space width using the positions of the anatomical landmarks. Finally, the processor can display the lower extremity image including a marker indicating the knee joint space width on a display device. The information of the knee joint space width obtained through this process may be provided to the patient or clinical physician as the result of automatic analysis.

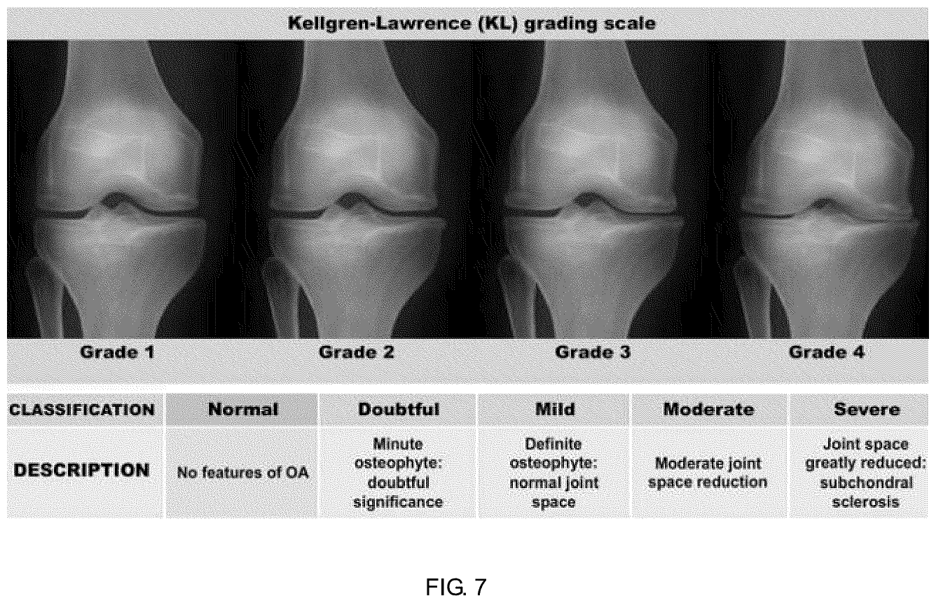

FIG. 7 shows the Kellgren-Lawrence grading scale used to experiment with the performance of an apparatus 100 according to embodiments of the present disclosure.

To verify the performance of the apparatus 100, multiple lower extremity images of osteoarthritis patients based on the Kellgren-Lawrence grading scale were retrospectively analyzed.

In experiments, 30 lower extremity images of osteoarthritis patients with a grade of 3 or 4 according to the Kellgren-Lawrence grading scale were used. Cases with severe deformities were excluded.

Digital reconstruction radiography (DRR) preprocessing images were generated by reconstructing the AP (anteroposterior, forward-backward) plane image of the knee from CT images taken with 0.6 mm intervals for each osteoarthritis patient at various beam projection angles. In this case, the beam projection angle for the posterior tibial slope (PTSA) in the AP plane was set to 20°, 10°, 5°, 0°, −5°, −10°, and −20°. Subsequently, the first distance (D1) of each osteoarthritis patient, the anterior border distance (distance of anterior border, DAB), and the second distance (D2), the posterior border distance (distance of posterior border, DPB), were each measured, and the average distance was derived.

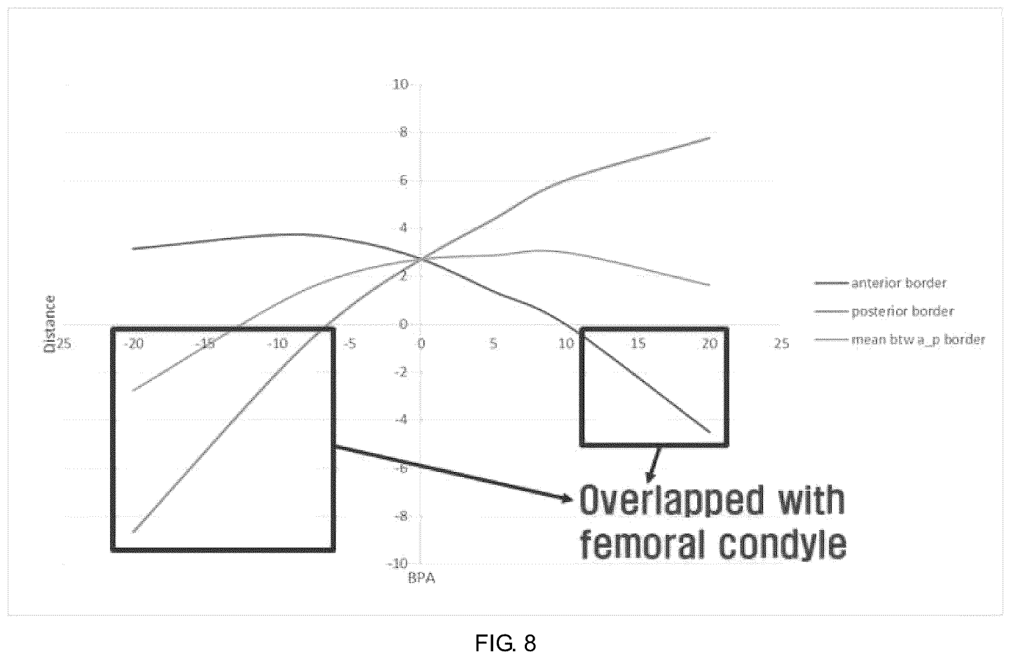

FIG. 8 is a graph showing the correlation between the first distance, the second distance, and the average distance analyzed by an apparatus according to embodiments of the present disclosure.

Referring to FIG. 8, the first distance (D1), the anterior border distance, shows a negative correlation with the beam projection angle (BPA). This means that as the beam projection angle increased, the anterior border distance decreased. In other words, the anterior border of the tibia (a) overlapped with the femoral condyle (f) when the beam projection angle was 10° or more.

In addition, the second distance (D2), the posterior border distance, shows a positive correlation with the beam projection angle (BPA). This means that as the beam projection angle increased, the posterior border distance increased. In other words, the posterior border of the tibia (p) overlapped with the femoral condyle (f) when the beam projection angle was less than −10°.

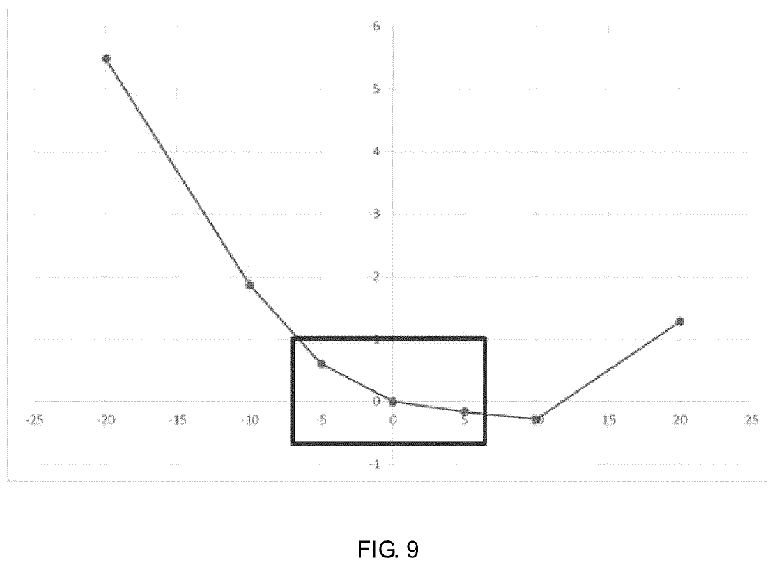

FIG. 9 is a graph showing a difference between an average distance at different beam projection angles (BPA) and an actual joint space width (JSW) analyzed by an apparatus according to embodiments of the present disclosure.

Referring to FIG. 9, the average value of the actual joint space width was 2.71±0.59 mm (2.0 mm-3.9 mm). The average distance was 2.86±0.71 mm (P=0.06) when the beam projection angle was 5°. This is the closest value to the average value of the actual joint space width. It was found that the beam projection angle with caudal tilting is more consistent with the actual joint space width than the beam projection angle with cephalic tilting.

In addition, the average distance was 2.99±1.16 mm (P=0.120) when the beam projection angle was 10°. At other beam projection angles, there were differences from the average value of the actual joint space width (P<0.05).

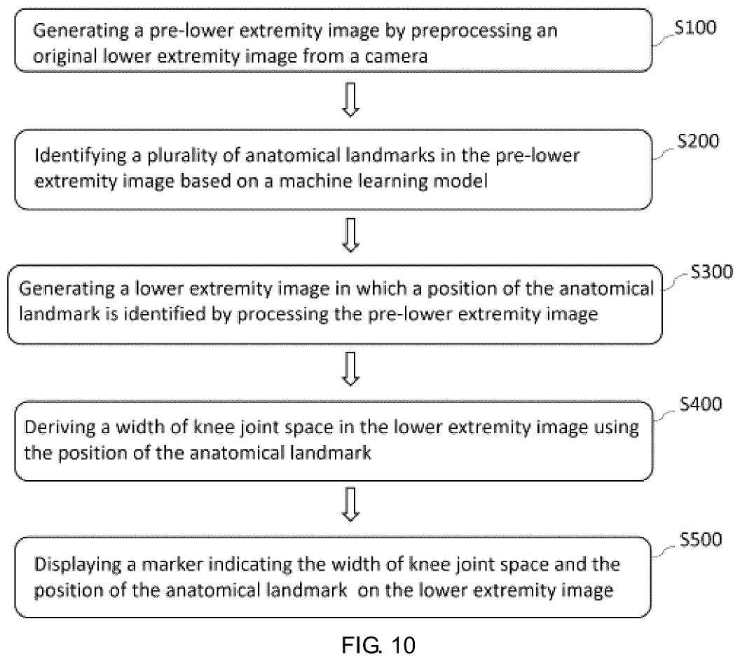

FIG. 10 shows a flowchart illustrating an exemplary process for automated analysis of a lower extremity image by a processor 111 according to embodiments of the present disclosure.

Referring to FIG. 10, at step S100, when an original lower extremity image is acquired from a camera or other device that is coupled to a computing device (as discussed in conjunction with FIG. 2), the processor 111 may preprocesses the original lower extremity image to generate a pre-lower extremity image. At step S200, the processor 111 may Identify a plurality of anatomical landmarks in the pre-lower extremity image based on a machine learning model. The plurality of anatomical landmarks may be included in at least one of hip joint image, knee joint image and ankle joint image enlarged in the pre-lower extremity image. At step S300, the processor 111 may generate a lower extremity image in which a position of the anatomical landmark is identified by processing the pre-lower extremity image. At step S400, the processor 111 may derive a width of knee joint space in the lower extremity image using the position of the anatomical landmark. The position of the anatomical landmark may be the medial or lateral femoral condyle (c) and the medial or lateral anterior border of tibia (r), measured from the midline of the medial plateau or the center point of the medial plateau, the medial or lateral posterior border of tibia (q), measured from the center point (j) of the medial plateau (as discussed in conjunction with FIGS. 6A to 6C). At step S500, the processor 111 may display a marker indicating the width of knee joint space and the position of the anatomical landmark on the lower extremity image. The marker may be, but is not limited to, point and line.

As such, the apparatus according to embodiments of the present disclosure can automate the entire process of analysis using a machine learning model to analyze the joint space width of the knee in lower extremity images, thereby obtaining objective and accurate results in a short period of time.

Embodiments of the present invention may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. One skilled in the art will recognize no computing system or programming language is critical to the practice of the present disclosure. One skilled in the art will also recognize that a number of the elements described above may be physically and/or functionally separated into sub-modules or combined together.

Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. An apparatus for automated analysis of knee joint space in a lower extremity image, comprising:
a processor; and
a memory including one or more sequences of instructions which, when executed by the processor, causes steps to be performed comprising:
generating a pre-lower extremity image by preprocessing an original lower extremity image from a camera;
identifying a plurality of anatomical landmarks in the pre-lower extremity image based on a machine learning model;
generating the lower extremity image in which a position of the anatomical landmark is identified by processing the pre-lower extremity image; and
deriving a width of the knee joint space in the lower extremity image using the position of the anatomical landmark.

2. The apparatus of claim 1, wherein the steps further comprises:
generating images in which a specific anatomical area of the lower extremity image is enlarged; and
displaying the images on a display device.

3. The apparatus of claim 2, wherein the images comprise at least one of a hip joint image, a knee joint image and an ankle joint image.

4. The apparatus of claim 1, wherein the steps further comprises:
generating a marker indicating the width of knee joint space on the lower extremity image; and
displaying the lower extremity image including the marker on a display device.

5. The apparatus of claim 1, wherein the position of the anatomical landmark comprises at least one of a medial or lateral femoral condyle, a medial or lateral anterior border of tibia measured from a midline of the medial plateau or a center point of the medial plateau, a medial or lateral posterior border of tibia measured from the center point of the medial plateau.

6. The apparatus of claim 1, wherein the width of the knee joint space is derived by calculating an average of a first distance and a second distance,
wherein the first distance is a distance between a medial or lateral femoral condyle and a medial or lateral anterior border of tibia measured from a midline of a medial plateau or a center point of the medial plateau, and the second distance is a distance between the medial or lateral femoral condyle and a medial or lateral posterior border of tibia, measured from the center point of the medial plateau.

7. A method for automated analysis of knee joint space in a lower extremity image, comprising, comprising:
generating a pre-lower extremity image by preprocessing an original lower extremity image from a camera;
identifying a plurality of anatomical landmarks in the pre-lower extremity image based on a machine learning model;
generating the lower extremity image in which a position of the anatomical landmark is identified by processing the pre-lower extremity image; and
deriving a width of the knee joint space in the lower extremity image using the position of the anatomical landmark.

8. The method of claim 7, further comprising:
generating images in which a specific anatomical area of the lower extremity image is enlarged; and
displaying the images on a display device.

9. The method of claim 7, further comprising:
generating a marker indicating the width of knee joint space on the lower extremity image; and
displaying the lower extremity image including the marker on a display device.

10. The method of claim 7, wherein the width of the knee joint space is derived by calculating an average of a first distance and a second distance,
wherein the first distance is a distance between a medial or lateral femoral condyle and a medial or lateral anterior border of tibia measured from a midline of a medial plateau or a center point of the medial plateau, and the second distance is a distance between the medial or lateral femoral condyle and a medial or lateral posterior border of tibia, measured from the center point of the medial plateau.
